(19) United States
(12) Patent Application Publication
(10) Pub. No.: US 2011/0112520 A1
(43) Pub. Date: May 12, 2011

(54) METHOD AND DEVICE FOR FAT TREATMENT

(75) Inventor: Kreindel MICHAEL, Richmond Hill (CA)

(73) Assignee: INVASIX CORPORATION, Richmond Hill (CA)

(21) Appl. No.: 12/616,421

(22) Filed: Nov. 11, 2009

(51) Int. Cl. A61B 18/14; A61B 18/18

(52) U.S. Cl. 606/13; 606/33; 606/41

(57) ABSTRACT

A method and apparatus for fat destruction and body contouring. The method comprises pre-heating treated tissue and applying a high voltage pulse to destroy adipose tissue. The device includes an energy source for tissue heating and an HV pulse generator for applying voltage pulsed in the range of 200V to 5 kV.

### FIELD OF THE INVENTION

The invention relates to methods and device for skin tightening and fat destruction.

### BACKGROUND OF THE INVENTION

Body sculpturing is a one of the most popular cosmetic procedures in the US.

Obesity is the number one fastest growing disease in the United States. Skin tissue consists of an outer epidermal layer overlying a dermal layer that is in contact with a layer of subcutaneous adipose tissue. Excess adipose tissue is responsible for such medical problems as obesity, cellulites, loose skin, and wrinkles. Localized collections of excess fat, focal lipodystrophies, result in convex skin distension and undesirable skin contours. By destroying the adipose cells, the appearance of the outer layer of the skin can be improved and the convex distensions reduced and the contour improved. Damaged adipose tissue is evacuated from the body by the lymphatic system. The destruction of adipose tissue in the sub-dermal layer often provides the following medical and cosmetic solutions: weight reduction, cellulite reduction, loose skin reduction, deep wrinkle reduction and body and face re-contouring. Reduction of fat in subcutaneous layer can create loose skin that should be tightened to create an aesthetically pleasing skin appearance.

Most existing skin tightening methods target the collagen but do not have a significant effect on adipose tissue. Radio frequency (RF) energy has been actively used for the treatment of dermal layers of the skin. For example U.S. Pat. No. 6,749,626 describes the use of RF energy for collagen formation in the dermis U.S. Pat. Nos. 6,470,216, 6,438,424, 6,430446 and 6,461,378 disclose methods and apparatuses for destroying the collagen matrix using RF, cooling and a special electrode structure that smoothes the skin surface. U.S. Pat. Nos. 6,453,202, 6,405,090, 6,381,497, 6,311,090, 5,871,524 and 6,425,912 describe methods and apparatuses for delivering RF energy to the skin using membrane structure. U.S. Pat. Nos. 6,453,202 and 6,425,912 describe method and apparatus for delivering RF energy to the skin using dielectric electrodes. U.S. Pat. Nos. 6,381,498, 6,377,855, 5,919,219, 5,948,011, 5,755,753 describe methods of collagen contraction using RF energy, and a reverse temperature gradient on the skin surface.

U.S. Pat. Nos. 6,378,380, 6,377,854 and 5,660,836 describe methods of liposculpturing using RF energy and external cooling to affect the collagen inside the adipose tissue.

Another method to reduce and redistribute adipose issue is skin massaging. This method is based on improving of blood circulation and increasing fat metabolism. U.S. Pat. No. 6,662,054 describes a method for skin massaging in combination with non-aggressive RF heating for increasing skin and fat metabolism.

U.S. Pat. No. 6,273,884 to Altshuler et al. discloses simultaneous application of optical energy and negative pressure to the skin in order to treat a skin defect. This method is limited by the light penetration depth, which does not exceed a 1-2 millimeters.

The above mentioned methods attempt to solve the problems created by localized excesses of adipose tissue such as body contouring, loose skin, and deep wrinkles, by contracting the superficial collagen tissue or directly altering the fat cell. These methods are limited in their penetration depth and by the degree of deep fat contouring that can be achieved by modest tightening of the skin. A more effective and longer lasting result would be achieved by directly targeting and destroying the adipose tissue. However, in order to reach the sub-dermal layer adipose tissue, it is necessary to deliver energy to the deeper layer of tissue.

U.S. Pat. No. 5,143,063 describes a method based on thermal destruction of fat using the focusing of microwave or ultrasound energy in adipose tissue. But both types of energy are very expensive and its safety limitations are not clear.

U.S. Pat. No. 6,047,215 describes device were two RF antennas are applied to the skin surface and directed to the same area inside the adipose tissue to create higher energy density inside the body than on the surface.

Another, non-thermal method of destruction of adipose tissue is electroporation. This method is based on irreversible change of cell membrane using high electrical field leading to apoptotic death of cells.

US application 20040019371 describes device and method comprising at least two electrodes and generating electrical pulses with voltage above electroporation threshold.

U.S. Pat. No. 6,697,670 describes device with two pairs of electrodes where first pair is used for electroporation of adipose tissue and second pair for electro-stimulation.

The limitations of electroporation are the use of high voltage (HV) that may create arcing around electrode and damage the skin surface. Using multiple pulses with a single polarity may create a risk of electrical shock. In addition, generation of short pulses with an amplitude of a few kilovolts has many technical challenges.

HV electrical pulses are also used for sterilization by destroying bacteria. In the article “Electrical Sterilization of Juice by Discharged HV Impulse Waveform”, Hee-Kyu Lee, American Journal of Applied Sciences 2 (10): 2076-2078, 2006 it is shown that survivability of the cells is strongly related to temperature and an increase in temperature from 30° C. to 40° C. may provide the same survivability at the half the electric field strength.

The article “Comparison of Electroporation Threshold for Different Cell Lines in vitro”, G. Saulis and R. Saule, Proceedings of the 2nd Euro-Asian Pulsed Power Conference, Vilnius, Lithuania, Sep. 22-26, 2008 demonstrates that for large cells the threshold of irreversible electroporation is strongly related to cell size. Thus, for cells with a 3 micron diameter the threshold is twice as high as for cells with 6-7 micron diameters. Fat cells of an adult person have a size in the range of 20 microns to 100 microns and should have an electroporation threshold, which is significantly lower. Pre-heating of adipose tissue prior the electroporation treatment reduces the treatment threshold to a level that is practical for use without risk for the treated patient and can be used for fat reduction and body counturing.

### SUMMARY OF THE INVENTION

The present invention provides a method of body countering by destroying fat cells using pulses of High Voltage (“HV”) electricity applied to tissue pre-heated by another energy source. Pre-heating of the tissue allows destroying cells at a much lower electric field strength than the electroporation threshold found under normal conditions. Higher sensitivity of larger cells to the electroporation allows one to selectively destroy only large cells with a size of 50 microns and larger. This method of selective treatment does not affect skin and organs while the largest adipocytes are irreversibly damaged.

In a first embodiment the tissue heating is done using Radio Frequency (“RF”) energy applied to the treated tissue. RF electrodes are attached to the skin surface or inserted into the body and RF current flowing through the tissue generates the required heat. A mono-polar configuration of RF electrodes may be used or a bi-polar configuration with two or more electrodes maybe applied to the area being treated. RF energy can be delivered in Continuous Wave (“CW”) or in burst mode. To create more uniform distribution over the treatment depth the electrodes may be cooled using cold liquid, thermo-electric coolers or forced air. The frequency range of RF current is typical for electrosurgical devices and is in the range of 0.3 MHz up to 50 MHz. Alternatively higher frequencies of electromagnetic energy in the microwave range may be used for tissue heating. Tissue impedance may be monitored to insure good electrode coupling to the tissue and proper RF energy delivery.

In an other embodiment, optical energy in the visible and/or infrared range may be used to pre-heat the tissue to be treated. Preferably, the near infrared part of the light spectrum may be used with a light penetration depth of more than 1 mm to introduce at least part of the optical energy below dermal layer. An incoherent light source as gas discharge or incandescent lamps may be used as a light source with filter optimizing light spectrum for better and safer heating. A laser may also be used for generating optical energy and tissue heating. A diode laser with wavelengths in the range of 600 nm to 2000 nm provides good efficiency of heating. Optical energy may be delivered in pulsed and CW mode to the tissue surface and into the tissue using optical fiber.

In yet another embodiment the tissue may be heated by heat transfer from a body having a heated surface in contact with the tissue. This method is very non-expensive due to it's simplicity but heat transfer to a depth of 1 cm can take a few minutes and significantly prolong treatment time. As a heat source, pre-heated liquid may be used or a heated metal plate connected to the energy source on one side and attached to the tissue on the other side.

In all three methods of heating the desired tissue temperature should not exceed 45° C. to avoid tissue thermal damage and pain. Tissue temperature may be monitored using a thermistor, thermocouple or other temperature sensor with the applied heating energy being stopped when desired heating is achieved

HV pulses delivered to the tissue should be in the range of 100 V/cm up to 5 kV/cm, that is, lower than the reported threshold of irreversible electroporation for cells at normal conditions. Pulse duration should be short enough to avoid electrical shocking. Another reason to keep a short pulse duration is to avoid air ionization. The appearance of a spark may burn the tissue surface. The preferable pulse duration is 1 ns to 10 microseconds. In order to increase efficiency of treatment, a burst of pulses may be applied with a delay between pulses long enough for recombination of ions and electrons in the air to reduce the risk of sparking.

To avoid electrical shocking, pulses of opposite polarity may be applied to tissue periodically or pulses of high frequency alternating voltage with a frequency of 100 kHz up to 100 mHz. At a high frequency of the alternating current the average current will be equal to zero and HV will be applied without risk of electrical shock or unpleasant sensation. HV pulses may be delivered through the same electrode electrodes as the RF energy or through separate electrodes.

Negative pressure may be used to attach tissue to the electrodes.

The system comprises the following main components:
A power supply that converts AC voltage from the wall plug to stabilized DC voltage.
A heat generator that connected to the power supply.
An HV pulse generator connected to the power supply.
   The system may have a controller that controls the RF parameters and a user interface including an LCD screen and a touch screen. The controller may have a microprocessor and dedicated software. A monitoring system is required to measure RF parameters including tissue impedance and/or RF current and/or RF voltage or other electronic parameters. The system has connector to connect hand piece with treatment cavity to the device.

In a preferred embodiment the HV pulses and RF energy are delivered through the same electrodes located in the cavity. The treatment is conducted with following steps:
The cavity is applied to the skin surface with it's opening facing the skin surface;
negative pressure is applied to the cavity to aspirate part of skin with fat into the cavity and couple it to the electrodes;
RF energy is applied to the tissue and the temperature of the skin surface is monitored during the heating;
when skin temperature reaches a predetermined value, one or more HV pulses are applied;
The negative pressure is released and the hand piece is moved to the new area.
   This invention can be used for body contouring, face lifting and cellulite treatment. For body treatment a larger cavity and higher heating power should be used while for face treatment a smaller hand piece and lower power are more suitable.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 is a sectional view showing a cavity with protruded skin, two electrodes delivering RF energy and HV pulses into the protruded tissue volume;

FIG. 2 is a schematic diagram of an apparatus according to the present invention;

FIG. 3 is a sectional view of an alternate embodiment showing a cavity with protruded skin, two electrodes delivering HV pulses into the protruded tissue volume and heating elements attached to electrodes; and,

FIG. 4 is a sectional view of a further alternate embodiment showing a cavity with protruded skin, two electrodes delivering HV pulses into the protruded tissue volume and lamps with reflectors for heating the protruded tissues;

### DETAILED DESCRIPTION OF PREFERRED EMBODIMENTS

The present invention provides a method of body counturing through fat cell destruction using pulses of HV applied to tissue pre-heated by another energy source. Pre-heating of the tissue allows destroying cells at much lower electric field strength than the electroporation threshold found for normal conditions. Higher sensitivity of larger cells to the electroporation allows selective destruction of only large cells with a size of 50 microns and larger. This method of selective treatment does not affect skin and organs while largest adipocytes are irreversibly damaged.

In a first embodiment the tissue heating is done using RF energy applied to the treated tissue. The RF electrodes are attached to the skin surface or inserted into the body and RF current flowing through the tissue generates required heat. Either a mono-polar configuration of RF electrodes or a bi-polar configuration with two or more electrodes applied to the treated area may be used. RF energy can be delivered in continuous wave (CW) or in burst mode.

To create more uniform distribution over the treatment depth the electrodes can be cooled using cold liquid, thermo-electric coolers or by forced air. The frequency range of RF current is typical for electrosurgical devices and is in the range of 0.3 MHz up to 50 MHz. Alternatively higher frequencies of electromagnetic energy in the microwave range can be used for tissue heating. Tissue impedance can be monitored to ensure good electrode coupling to the tissue and proper RF energy delivery.

In another embodiment, optical energy in the visible and/or infrared range can be used to pre-heat the treated tissue. Preferably, the near infrared part of light spectrum with a light penetration depth of more than 1 mm should be used to introduce at least part of the optical energy below dermal layer. The light source may for example be a gas discharge or incandescent lamp with a filter for optimizing the light spectrum for better and safer heating. A laser can also be used for generating optical energy and tissue heating. For example a diode laser emitting light having a wavelength in the range of 600 nm to 2000 nm provides good efficiency of heating. Optical energy can be delivered in pulsed and CW mode to the tissue surface and into the tissue using optical fiber.

In yet another embodiment the tissue can be heated by heat transfer from a heat source attached to the tissue. This method is very inexpensive due to its simplicity but heat transfer to a depth of 1 cm can take a few minutes and prolong significantly the treatment time. By way of example, as a heat source pre-heated liquid can be used or a metal plate connected to an energy source on one side and attached to the tissue on the other side.

In all three methods of heating the desired tissue temperature should not exceed 45° C. to avoid tissue thermal damage and pain. Tissue temperature can be monitored using a thermistor, thermocouple or other temperature sensor with the applied heating energy being stopped when a desired heating is achieved.

HV pulses delivered to the tissue should be in the range of 100 V/cm up to 5 kV/cm that is lower than reported threshold of irreversible electroporation for cells at normal conditions. Pulse duration should be short enough to avoid electrical shocking. Another reason for short pulse duration is to avoid air ionization. The appearance of sparks may burn tissue surface. The preferable pulse duration is 1 ns to 10 microseconds. In order to increase efficiency of treatment a burst of pulses can be applied with a delay between pulses long enough for recombination of ions and electrons in the air to reduce the risk of sparking.

To avoid electrical shocking, pulses of opposite polarity can be applied to tissue periodically or the pulses may be of a high frequency alternating voltage with a frequency of from 100 kHz up to 100 mHz. At a high frequency of alternating current the average current will be equal to zero and HV will be applied without risk of electrical shock or unpleasant sensation. HV pulses can be delivered through the same electrode or electrodes as RF energy or through separate electrodes.

Negative pressure can be used to attach tissue to the electrodes.

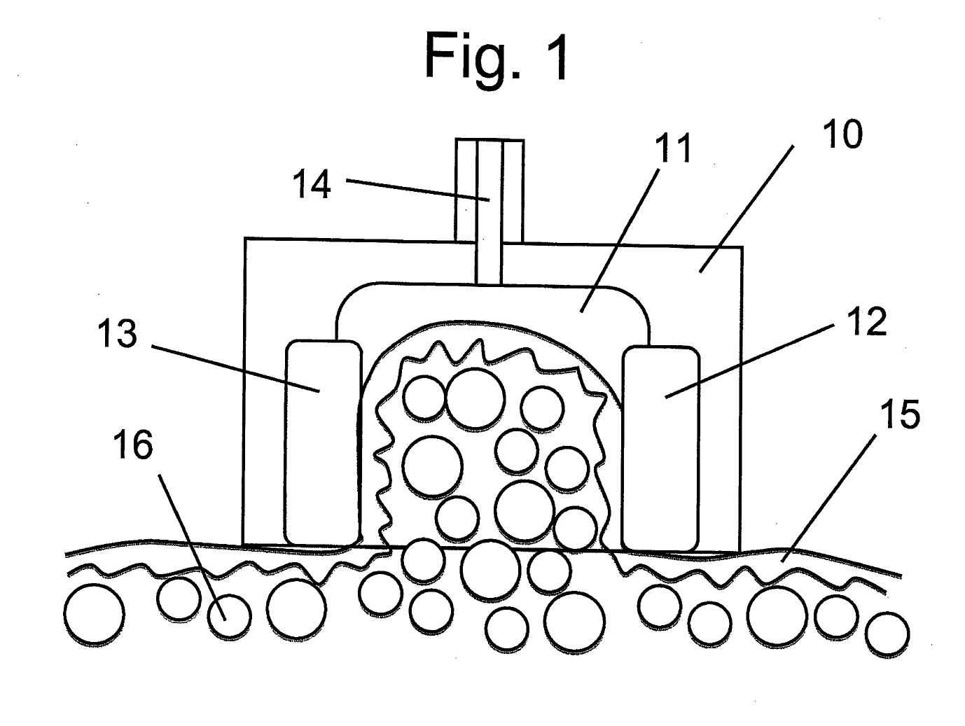

Referring to FIG. 1, a hand piece assembly is shown which comprises a housing 10 having a cavity 11. Two RF electrodes 12, 13 are mounted within the cavity 11. Negative pressure applied through the inlet 14 pulls skin 15 and subcutaneous fat 16 into the cavity 11 and causes the skin to contact the electrodes 12,13. This pair of electrodes 12, 13 delivers to the skin 15 and fat 16 an RF energy and high voltage pulses. The arrangement localizes RF energy and electric field generated by the HV pulse to the limited volume inside the cavity reducing the risk of accidental damage to other tissue.

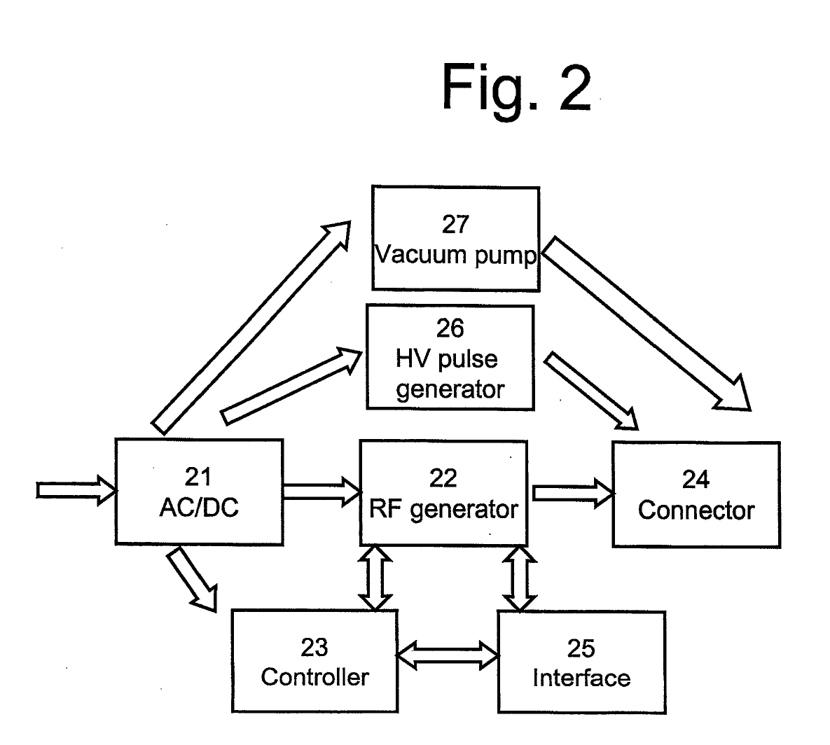

FIG. 2 schematically illustrates the generation and control unit for generating RF energy and HV pulses for delivery through the RF electrodes. The unit comprises a power supply 21 for converting AC voltage to the stabilized DC voltage in the range of 12VDC to 400 VDC. An RF generator 22 converts DC voltage supplied by the power supply 21 to a high frequency voltage in the frequency range of 0.2 MHz to 100 MHz. The preferable frequency range is 0.5 MHz to 10 MHz. RF energy can be delivered in CW or pulse mode.

The required average RF power will depend on electrode geometry and may be varied from 1 W up to 300 W. The preferable range of RF power is 10 W to 100 W. RF energy is delivered to a connector 24 of the unit which communicates with the RF electrodes 12, 13. One or more RF electrodes may be connected to the unit connector 24. An HV pulse generator 26 converts DC voltage from the power supply 21 into HV pulses delivered through the connector 24 to the same electrodes in the hand piece. A Controller 23 is used to control output from RF generator and HV pulse generator 26. The controller 23 provides communication between a user interface 25 and electronics inside the unit. The controller 23 receives inputs from an operator through the user interface 25 and adjusts the output RF and HV pulse parameters according to the inputs.

A Vacuum pump 27 may be connected to the hand piece cavity 11 to create the negative pressure for drawing tissue to be treated against the electrodes.

In using the apparatus and method of the present invention to treat subcutaneous adipose tissue and tighten skin, the following exemplary parameter values of RF energy may be used:
RF frequency: 0.2-100 MHz.
Average output power: from about 1 to about 300 W.
Delivered energy should create a high enough temperature in the tissue to allow fat cell destruction by HV pulses in the range of 200V up to 5 kV.

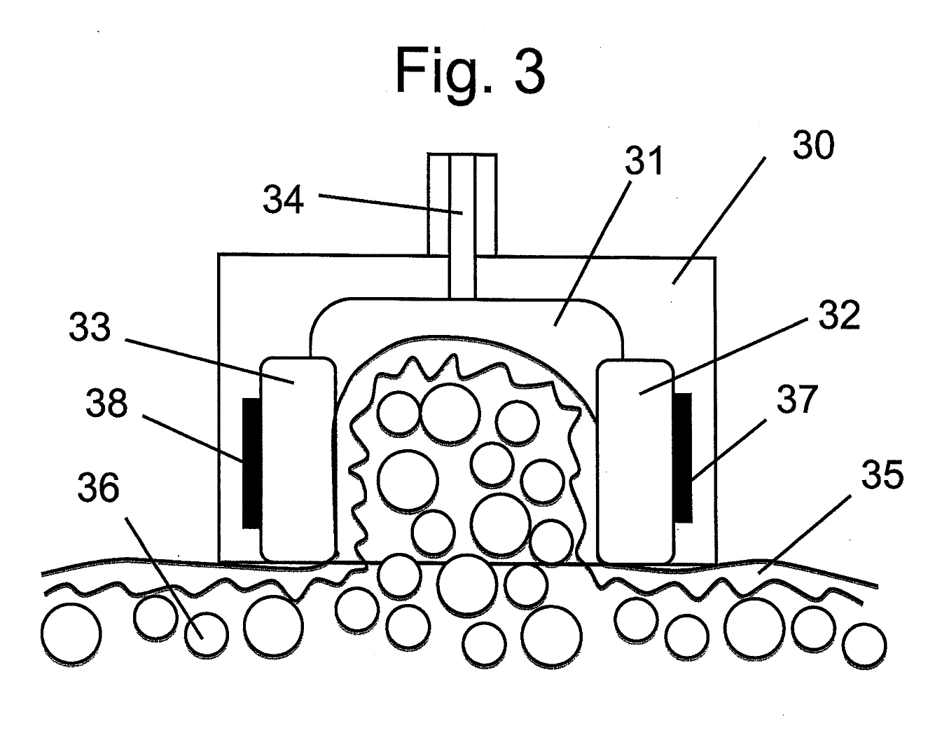

Referring to FIG. 3, a hand piece assembly is shown comprising a housing 30 with a cavity 31. Two electrodes 32, 33 are assembled inside the cavity 31. Negative pressure applied through the inlet 34 pulls skin 35 and subcutaneous fat 36 into the cavity 31 causing the skin to contact the electrodes 12,13.

This pair of electrodes 32, 33 are heated by respective heating elements 37, 38 and deliver to the skin 15 and fat 16 high voltage pulses. The arrangement localizes heat transferred from electrodes 32, 33 and the electric field generated by the HV pulse in a limited volume inside the cavity reducing the risk of accidental damage to other tissue.

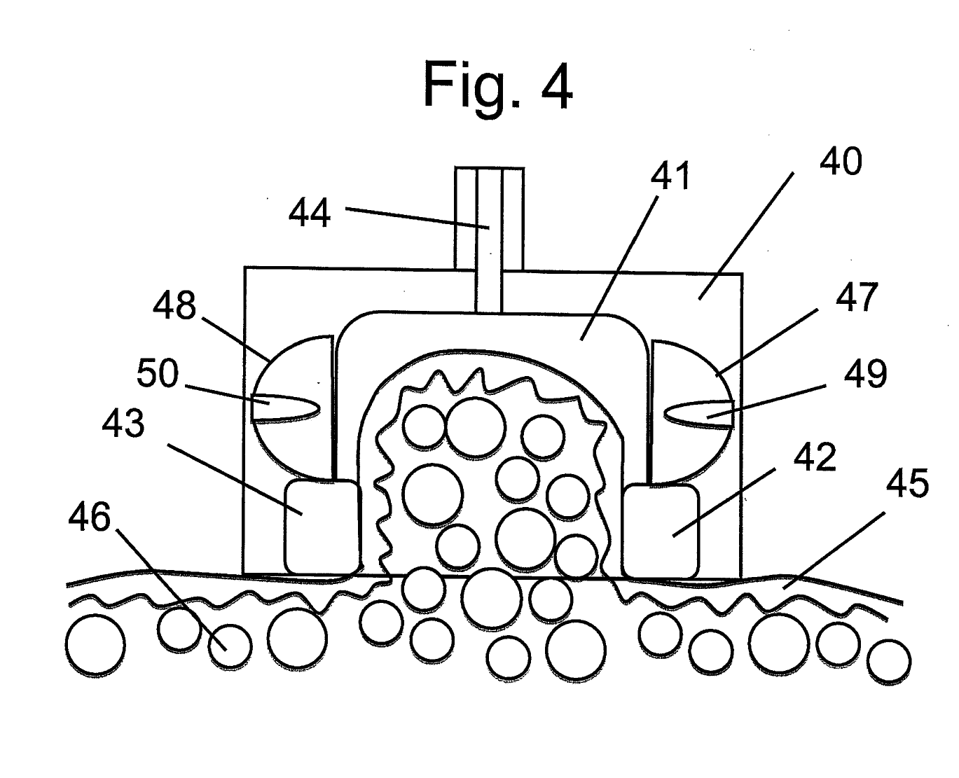

Referring to FIG. 4, a handpiece assembly is shown comprising a housing 40 with a cavity 41. Two electrodes 42, 43 are assembled inside the cavity 41. Negative pressure applied through the inlet 44 pulls skin 45 and subcutaneous fat 46 into the cavity 41 skin to contact the electrodes 42, 43.

This pair of electrodes 42, 43 delivers to the skin 45 and fat 46 high voltage pulses. Light emitted by two lamps 49, 50 is directed to the tissue by reflectors 47, 48 to preheat the skin prior to applying the HV pulse.

This arrangement localizes heat created by optical energy from the lamps 49, 50 and the electric field generated by the HV pulse in a limited volume inside the cavity reducing the risk of accidental damage to other tissue.

The above description has been described by way of example. It will be apparent to persons skilled in the art that a number of variations and modifications may be made without departing from the scope of the invention as defined in the claims.

1. An apparatus for fat destruction and body contouring comprising:
at least two electrodes coupleable to a body area to be treated;
an RF generator connected to the electrodes for delivering RF energy to the treated tissue to elevate tissue temperature above a normal level; and,
a high voltage (HV) pulse generator connected to the electrodes for delivering to the pre-heated tissue at least one high voltage pulse in the range of from 200V to 5 kV to cause selective destruction of fat cells wherein the RF and HV pulse generators are respectively connected to common or different of the at least two electrodes.

2. An apparatus according to claim 1 wherein the electrodes are mounted within a cavity, and said cavity fluidly communicates with a vacuum source for drawing the body area to be treated into the cavity and against the electrodes thereby coupling to the body area to be treated to the electrodes using negative pressure.

3. An apparatus according to claim 1 wherein the HV pulse duration is in the range from 1 ns to 10 microseconds.

4. An apparatus according to claim 1 wherein the RF current is generated in the frequency range of 0.2 MHz to 100 MHz.

5. An apparatus according to claim 1 wherein the RF and HV pulse generators are respectively coupled to at least a common one of the at least two electrodes.

6. An apparatus for fat destruction and body contouring comprising:
at least two electrodes for coupling to the body area to be treated;
a light source mountable adjacent to the area to be treated for delivering light energy to the treated tissue to elevate tissue temperature above a normal level; and,
a high voltage (HV) pulse generator connected to the electrodes for delivering to the pre-heated tissue at least one high voltage pulse in the range of from 200V to 5 kV to cause selective destruction of fat cells.

7. An apparatus according to claim 6 wherein the electrodes are mounted within a cavity, and said cavity fluidly communicates with a vacuum source for drawing the body are to be treated into the cavity and against the electrodes thereby coupling to the body area to be treated to the electrodes using negative pressure.

8. An apparatus according to claim 6 wherein the HV pulse duration is in the range from 1 ns to 10 microseconds.

9. An apparatus according to claim 6 wherein the light source is a lamp or a laser.

10. An apparatus for fat destruction and body contouring comprising:
at least two electrodes coupleable to the body area to be treated; and,
a pre-heated surface coupleable to the treated area to elevate tissue temperature above a normal level; and,
a high voltage (HV) pulse generator connected to the electrodes for delivering to the pre-heated tissue at least one high voltage pulse of voltage in the range of from 200V to 5 kV to cause selective destruction of fat cells.

11. An apparatus according to claim 10 wherein the electrodes are mounted within a cavity, and said cavity fluidly communicates with a vacuum source for drawing the body area to be treated into the cavity and against the electrodes thereby coupling to the body area to be treated to the electrodes using negative pressure.

12. An apparatus according to claim 10 wherein the HV pulse duration is in the range from 1 ns to 10 microseconds.

13. An apparatus according to claim 10 wherein the pre-heatable surface is heatable to a temperature above 40 degrees centigrade.

14. An apparatus according to claim 1 wherein the RF and HV pulse generators are respectively coupled to at least a common one of the at least two electrodes.

15. A method for thermal fat destruction and body contouring comprising the steps of:
i) coupling at least two electrodes to the tissue to be treated;
ii) pre-heating the tissue to be treated to a temperature above its normal temperature; and,
iii) applying to the pre-heated tissue at least one pulse of voltage in the range of from 200V up to 5 kV to cause selective destruction of the fat.

16. A method according to claim 15 where tissue is pre-heated using RF energy.

17. A method according to claim 15 where tissue is pre-heated using light energy.

18. A method according to claim 15 where tissue is pre-heated using contact with a surface having temperature higher than normal tissue temperature.

19. A method according to claim 15 where tissue is coupled to the electrodes by negative pressure.

20. Use of the method according to claim 15 in a process selected from the group consisting of:
reducing body weight;
local fat reduction;
cellulite reduction;
face lifting; and,
body surface tightening.
